(19) United States
(12) Patent Application Publication
(10) Pub. No.: US 2024/0325502 A1
(43) Pub. Date: Oct. 3, 2024

(54) PROTEIN NANOSPHERES TO TREAT RENAL FAILURE, DYSFUNCTION OR DAMAGE

(71) Applicant: Richard C.K. Yen, Yorba Linda, CA (US)

(72) Inventor: Richard C.K. Yen, Yorba Linda, CA (US)

(21) Appl. No.: 18/735,523

(22) Filed: Jun. 6, 2024

Related U.S. Application Data
(63) Continuation-in-part of application No. 17/094,114, filed on Nov. 10, 2020.
Application No. 17/094,114 is a continuation-in-part of application No. 15/233,779, filed on Aug. 10, 2016, now Pat. No. 11,260,109.
Application No. 15/233,779 is a continuation-in-part of application No. 16/505,257, filed on Jul. 8, 2019, now Pat. No. 11,260,110.
Application No. 16/505,257 is a continuation-in-part of application No. 14/953,066, filed on Nov. 27, 2015, now abandoned.
(63) Continuation-in-part of application No. 17/208,736, filed on Mar. 22, 2021.
Application No. 17/208,736 is a continuation-in-part of application No. 15/233,779, filed on Aug. 10, 2016, now Pat. No. 11,260,109.
Application No. 15/233,779 is a continuation-in-part of application No. 16/505,257, filed on Jul. 8, 2019, now Pat. No. 11,260,110.
Application No. 16/505,257 is a continuation-in-part of application No. 17/094,114, filed on Nov. 10, 2020.
(63) Continuation-in-part of application No. 18/295,829, filed on Apr. 4, 2023.
Application No. 18/295,829 is a continuation-in-part of application No. 17/094,114, filed on Nov. 10, 2020.
Application No. 17/094,114 is a continuation-in-part of application No. 17/208,736, filed on Mar. 22, 2021.
(60) Provisional application No. 63/628,513, filed on Jul. 31, 2023.
(60) Provisional application No. 63/630,952, filed on Mar. 18, 2024.
(60) Provisional application No. 63/100,670, filed on Mar. 25, 2020.
(60) Provisional application No. 63/372,883, filed on Apr. 13, 2022.

(51) Int. Cl. A61K 38/36; A61K 9/16; A61P 13/12

(52) U.S. Cl. CPC A61K 38/363; A61K 9/1658; A61P 13/12

(57) ABSTRACT

A product and method of using albumin nanoparticles for treating renal failure, dysfunction or damage in a subject by administering a therapeutically effective amount of an albumin nanoparticle suspension containing fibrinogen coated albumin spheres (FAS) to increase a concentration of stem cells or precursors cells in their original site and/or augment a function or effectiveness of them in vivo to mobilize these stem cells or the precursor cells toward a trauma site. The renal failure, dysfunction or damage can be caused by administration of a renal-harmful agent or a chemotherapy drug. The suspension can be administered prophylactically and prior to administration of the agent or drug. The method can include the testing for biological markers, such as osteopontin, in the urine of the subject, and using the concentration information of the biomarkers to predict dosage of the suspension that is effective in further treatment.

### CROSS-REFERENCE TO RELATED APPLICATION

This application claims the benefit of priority under 35 U.S.C. § 119(e) based upon co-pending U.S. provisional patent application Ser. No. 63/628,513 filed on Jul. 31, 2023, and U.S. provisional patent application Ser. No. 63/630,952 filed on Mar. 18, 2024. The entire disclosure of the prior provisional applications are incorporated herein by reference.

This application is a continuation-in-part under 35 U.S.C. § 120 based upon co-pending U.S. patent application Ser. No. 17/094,114 filed on Nov. 10, 2020, co-pending U.S. patent application Ser. No. 17/208,736 filed on Mar. 22, 2021, and co-pending U.S. patent application Ser. No. 18/295,829 filed on Apr. 4, 2023. The entire disclosure of the prior non-provisional applications are incorporated herein by reference in their entirety.

### BACKGROUND

### Technical Field

In some aspects, the present technology relates to a protein nanospheres to treat renal failure, dysfunction or damage for use in connection with mobilizing stem cells to treat acute and chronic renal failures. In some other aspects, the present technology relates to a method of treating renal failure, dysfunction or damage by administering a therapeutically effective amount of an albumin nanoparticle suspension containing fibrinogen coated albumin spheres (FAS) to increase the concentration of stem cells or precursors cells in their original site and/or augment a function or effectiveness of stem cells or precursor cells in vivo to mobilize these stem cells or the precursor cells toward a trauma site. In still some other aspects, the present technology relates to a method of prophylactically treating renal failure, dysfunction or damage in a subject caused by administration of a renal toxic agent or chemotherapy drug.

### Background Description

Renal failure can be divided into acute and chronic renal failure. Treatment for acute renal failure depends mainly on removal or reduction of causes for the renal failure, e.g. cessation of antibiotics known to cause kidney failure. Treatment for chronic renal failure includes the use of “kidney dialysis” which are mechanical means of reducing the toxic material in the blood which are normally handled by functional kidneys.

Recent discoveries in stem cell therapies encouraged the use of stem cells obtained from the patient or from eligible donors. However, the results are equivocal and the expenses are high. Also, there is the real risk of the injected stem cells developing into tumors in the host. There is a need for new therapy involving the mobilization of the patient's own stem cells towards the mitigation of renal failure while avoiding the known problems of using “stem cells” from sources other than the patient himself or herself.

Yen has disclosed data showing that certain protein nanoparticles or nanospheres can be administered via the intravenous route, which will result in the attachment of such nanoparticles to the cells residing in the endothelium of the blood vessels. In addition, the same nanospheres called “Fibrinogen-coated albumin spheres (FAS)” can attach to bone marrow cells inside the bone marrow. Furthermore, administration of FAS intravenously has been shown to increase CD34+(stem) cells inside the bone marrow, followed by increased concentrations of certain “more mature” cells in the peripheral blood. The prior disclosures include a non-provisional patent application titled “Protein Nanospheres to Treat Harm from Multiple Trauma.” The application claims the benefit of priority under 35 U.S.C. § 119(e) based upon co-pending U.S. provisional patent application Ser. No. 63/372,883 filed on Apr. 13, 2022. The application is a continuation-in-part under 35 U.S.C. § 120 based upon co-pending U.S. non-provisional patent application Ser. No. 17/208,736 filed on Mar. 22, 2021, and co-pending U.S. non-provisional patent application Ser. No. 17/094,114 filed on Nov. 10, 2020.

Specifically, a dose of Fibrinoplate-S (FPS, the name of the drug product) administered intravenously has been shown to result in the entry of the active drug substance called fibrinogen-coated albumin spheres (FAS) into the bone marrow compartment of the bone marrow. In some experiments, the FAS can be pre-labeled with a fluorescent compound called Fluorescein IsoThioCyanate (FITC). Following isolation of bone marrow cells from the bone marrow, Fluorescein-labeled FAS can be seen to still attach to the isolated bone marrow cells. In separate experiments, unlabeled (original) FAS administered intravenously to animals have been shown to result in an increase of CD34+ cells in the bone marrow. Subsequently, an increased concentration of CD34+ cells can also be detected in the blood. As for a deep wound, as late as weeks after the administration of FPS, CD34+ cells can still be detected at the destination (resulting in the rapid healing of the wound). Such deep wounds include wounds caused by high dose of local irradiation to the skin (“irradiation-induced skin injury”.) However, there was no data presented yet to show that FPS can mobilize stem cells which can be beneficial for the treatment of renal failure.

Given the ineffectiveness of various prior art to cure renal failure, a less intrusive and more effective method of using stem cells from the patient to mitigate the harmful effects of renal failure and to cure the problem is needed

### SUMMARY

In view of the foregoing disadvantages inherent in the known types of stem cell therapies at least some embodiments of the present technology provides a novel protein nanospheres to treat renal failure, dysfunction or damage, and overcomes one or more of the mentioned disadvantages and drawbacks of the prior art. As such, the general purpose of at least some embodiments of the present technology, which will be described subsequently in greater detail, is to provide a new and novel protein nanospheres to treat renal failure, dysfunction or damage which has all the advantages of the prior art mentioned herein and many novel features that result in a protein nanospheres to treat renal failure, dysfunction or damage which is not anticipated, rendered obvious, suggested, or even implied by the prior art, either alone or in any combination thereof.

According to one aspect, the present technology can include a method of treating renal failure, dysfunction or damage in a subject in need thereof. The method can include administering a therapeutically effective amount of an albumin nanoparticle suspension containing fibrinogen coated albumin spheres (FAS) into the subject to increase the concentration of stem cells or precursors cells in their original site and/or augment a function or effectiveness of stem cells or precursor cells in vivo to mobilize these stem cells or the precursor cells toward a trauma site.

According to another aspect, the present technology can include a method of prophylactically treating renal failure, dysfunction or damage in a subject caused by administration of a renal toxic agent or chemotherapy drug. The method can include administering, prior to administration of the renal toxic agent or the chemotherapy drug, a therapeutically effective amount of an albumin nanoparticle suspension containing fibrinogen coated albumin spheres (FAS) into the subject to any one of or any combination of increase the concentration of stem cells or precursors cells in their original site, augment a function or effectiveness of the stem cells or precursor cells in vivo, and to mobilize the stem cells or the precursor cells toward a trauma site to mitigate harmful effects from the renal toxic agent or the chemotherapy drug.

According to still another aspect, the present technology can include an albumin nanoparticle suspension used for treating renal failure, dysfunction or damage in a subject in need thereof. The suspension can contain fibrinogen coated albumin spheres (FAS), and it can be configured any one of or any combination of increase the concentration of stem cells or precursors cells in their original site, augment a function or effectiveness of the stem cells or precursor cells in vivo, and to mobilize the stem cells or precursor cells toward a trauma site. A therapeutically effective amount of the suspension can be configured or configurable for intravenous administration into the subject.

In some embodiments, the renal failure, dysfunction or damage can be caused by administration to the subject of any one of or any combination of a renal-harmful agent, and a chemotherapy drug.

In some embodiments, the chemotherapy drug can contain platinum or can be CisPlatin.

In some embodiments, the chemotherapy drug is selected from the group consisting of Carboplatin (Paraplatin, Paraplatin AQ), Ifosfamide (Ifex), Methotrexate, Paclitaxel, Doxorubicin, and Gemcitabine.

In some embodiments, the therapeutically effective amount can be administered to the subject prophylactically and prior to administration of the renal-harmful agent or the chemotherapy drug.

In some embodiments, the stem cells or the precursor cells can be CD34+ stem cells.

In some embodiments, the fibrinogen coated albumin spheres can be configured to increase the concentration of CD34+ stem cells or precursors cells in their origin site and/or augment a function or an effectiveness of the CD34+ stem cells in vivo to mobilize these CD34+ stem cells toward a site of injury.

In some embodiments, the fibrinogen coated albumin spheres can be configured to increase cell populations of the CD34+ stem cell inside any one of or any combination of bone marrow, kidney, and a muscle.

In some embodiments, the fibrinogen coated albumin spheres can be configured to increase in cell populations positive for CD34 cell markers or negative for CD34 cell markers.

In some embodiments, the fibrinogen coated albumin spheres can be configured to produce a favorable anti-oxidative status within an injured tissue of the subject leading to accelerated recovery of the injured tissue.

In some embodiments, the fibrinogen coated albumin spheres can be configured to accelerate a rate of improvement of histological structures inside a kidney or injured muscles of the subject, leading to improved function of the kidney or the muscles.

In some embodiments, the fibrinogen coated albumin spheres can be configured to produce biological molecules or vesicles that have paracrine effects, leading to an accelerated rate of improvement of renal function or of histological structures inside a kidney or injured muscles of the subject.

In some embodiments, the subject can be human.

In some embodiments, the therapeutically effective amount can be 4 mg/kg or more administered to the subject intravenously.

In some embodiments, the therapeutically effective amount can be 8 mg/kg or more administered to the subject intravenously.

In some embodiments, the therapeutically effective amount can be administered to the subject prophylactically and prior to the renal failure, dysfunction or damage.

Some embodiments of the present technology can include a step of testing for one or more biological markers in urine of the subject.

In some embodiments, the one or more biological markers can be osteopontin.

In some embodiments, a concentration amount of the one or more biological markers can be configured to predict a dose of the albumin nanoparticle suspension which is effective in a further treatment of renal failure, dysfunction or damage in the subject.

In some embodiments, the renal failure, dysfunction or damage can be selected from the group consisting of diabetic renal failures, Autosomal Dominant Polycystic Kidney Disease (ADPKD), and Minimal Change Disease (MCD).

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other compositions, suspension, formulations and methods for carrying out the several purposes of the present technology. It is, therefore, that the claims be regarded as including such equivalent constructions insofar as they do not depart from the spirit and scope of the present technology.

It is therefore an object of the present technology to provide a new and novel protein nanospheres to treat renal failure, dysfunction or damage that has all of the advantages of the prior art stem cell therapies and none of the disadvantages.

It is another object of the present technology to provide a new and novel protein nanospheres to treat renal failure, dysfunction or damage that may be easily and efficiently manufactured, administered and marketed.

An even further object of the present technology is to provide a new and novel protein nanospheres to treat renal failure, dysfunction or damage that has a low cost of manufacture with regard to both materials and labor, and which accordingly is then susceptible of low prices of sale to the consuming public, thereby making such protein nanospheres to treat renal failure, dysfunction or damage economically available to the buying public.

Still another object of the present technology is to provide a new protein nanospheres to treat renal failure, dysfunction or damage that provides in the apparatuses and methods of the prior art some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof, with phantom lines (long-short-short-long lines) depicting environmental structure and forming no part of the claimed present technology. Such description makes reference to the annexed drawings wherein:

FIG. 1 is a reproduction of FIG. 1 from the Korrapati et al reference.

FIG. 2 is a graphical view of the results from the blood urea nitrogen (BUN) test on various days pre-CisPlatin or post-CisPlatin administration. It should be pointed out that various renal function tests such as BUN, Creatinine, Creatine Kinase are tests for the physiological, functional ability (normal or abnormal) of the kidney, but they are not necessarily reflective of any structural damages to the kidney. Physiological parameters can be easily affected by physiological conditions such as dehydration or over-hydration of the animal.

FIG. 3 is a graphical view of the results from the Creatinine test on various days pre-CisPlatin or post-CisPlatin administration.

FIG. 4 is a graphical view of the results from the Creatine Kinase test on various days pre-CisPlatin or post-CisPlatin administration.

FIG. 5 is a graphical view of the results from the Potassium test on various days pre-CisPlatin or post-CisPlatin administration.

FIG. 6 is a graphical view of the results from the Sodium test on various days pre-CisPlatin or post-CisPlatin administration.

FIG. 7 is a graphical view of the results from the Chloride test on various days pre-CisPlatin or post-CisPlatin administration.

FIG. 8 is a graphical view of the mean urine OPN concentration in each group from experiment 2 of the present technology. It should be pointed out that OPN is a sensitive biomarker which is very helpful to indicate any structural damage to the kidney, particularly the renal tubules.

The same reference numerals refer to the same parts throughout the various figures.

### DETAILED DESCRIPTION OF THE EMBODIMENTS

While the above-described devices fulfill their respective, particular objectives and requirements, the aforementioned devices or systems do not describe a protein nanospheres to treat renal failure, dysfunction or damage that allows mobilizing stem cells to treat acute and chronic renal failures. The present technology additionally overcomes one or more of the disadvantages associated with known methods and/or products by mobilizing stem cells to treat acute and chronic renal failures.

A need exists for a new and novel protein nanospheres to treat renal failure, dysfunction or damage that can be used for mobilizing stem cells to treat acute and chronic renal failures. In this regard, the present technology substantially fulfills this need. In this respect, the protein nanospheres to treat renal failure, dysfunction or damage according to the present technology substantially departs from the conventional concepts and designs of the prior art, and in doing so provides an apparatus primarily developed for the purpose of mobilizing stem cells to treat acute and chronic renal failures.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

Within the present description, the term “failure” refers to both “failure to function properly due to structural damage” (to be called “Damage”) and “failure to function properly due to physiological changes not necessarily traceable to structural injury or damage” (to be called “Dysfunction”.)

The term “kidney failure” refers to a condition where the dysfunction or damage is severe enough that the usual laboratory tests called “renal function tests” (measurement of the concentration of BUN, Creatinine etc. in the blood) will show abnormal values reflecting a “failure to perform the renal functions necessary for healthy life.”

Due to the fact that most “renal function tests” are not particularly sensitive, it is possible that a person suffering from some renal dysfunction or even damage may not show abnormal values associated with renal failure. More sensitive tests are needed (to be discussed below) to reveal renal damage which may or may not lead to renal failure, whether it is a temporary or permanent failure.

A review of the method of using stem cells for kidney failures has been published: “Stem Cell Therapies in Kidney Diseases: Progress and Challenges” authored by Rota et al and published in Int J Mol Sci. 2019 June; 20(11): 2790. The authors stated, “We summarize the renoprotective potential of pluripotent and adult stem cell therapy in experimental models of acute and chronic kidney injury and we explore the different mechanisms at the basis of stem cell-induced kidney regeneration.” The authors concluded that “The promise of stem cell therapies in pre-clinical models of kidney diseases is yet to be translated into more persuasive proof of clinical efficacy. Several clinical trials have confirmed the safety and tolerability of stem cells, and in particular of Mesenchymal Stem Cell, MSC-based therapies, in patients with renal diseases and kidney transplants. However, long-term monitoring is recommended to rule out the potential risk of cancer and of developing anti-HLA antibodies. Furthermore, the heterogeneity of MSCs obtained from different tissues and the lack of standardized protocols for isolation and in vitro culture are potential confounding variables that make the comparison of different clinical trials difficult, as highlighted recently.” Therefore, there is a distinct advantage, as disclosed in this present technology, to having the patient as the source of his or her own stem cells which are specific for the mitigation or cure of his or her own renal disease.

Another publication in Stem Cells Transl Med. 2017 February; 6(2): 405-418, titled “Clinical-Grade Isolated Human Kidney Perivascular Stromal Cells as an Organotypic Cell Source for Kidney Regenerative Medicine” authored by Daniëlle G. Leuning et al disclosed the importance of “perivascular stromal cells.” The authors explained, “Mesenchymal stromal cells (MSCs) are immunomodulatory and tissue homeostatic cells that have shown beneficial effects in kidney diseases and transplantation. Perivascular stromal cells (PSCs) identified within several different organs share characteristics of bone marrow-derived MSCs (BM-MSCs). These PSCs may also possess tissue-specific properties and play a role in local tissue homeostasis.”

Therefore, the authors hypothesized that “human kidney-derived PSCs (hkPSCs) would elicit improved kidney repair in comparison with BM-MSCs.” The authors introduce a novel, clinical-grade isolation method of hkPSCs from cadaveric kidneys by enriching for the perivascular marker, NG2. These hkPSCs show strong transcriptional similarities to BM-MSCs but also show organotypic expression signatures, including the HoxD10 and HoxD11 nephrogenic transcription factors.” The authors continued, “Comparable to BM-MSCs, hkPSCs showed immunosuppressive potential and, when cocultured with endothelial cells, vascular plexus formation was supported, which was specifically in the hkPSCs accompanied by an increased NG2 expression.” This research showed that PSCs obtained from kidneys (even from cadaveric kidneys) may be more effective than stem cells obtained from bone marrows.

It should be pointed out that Yen had already disclosed that the FAS from FPS has been seen to attach to endothelial cells, by the use of 2-photon microscopy to observe the interior of blood vessels in a live animal. (FIG. 4B of “Fibrinogen-Coated Albumin Nanospheres Prevent Thrombocytopenia-Related Bleeding” by Sung et al, published in Radiat Res. 2020 Aug. 1; 194(2): 162-172.) However, more studies need to be conducted to show whether these endothelial cells capable of FAS attachment are similar to, or different from the MSC, or hkPSC described in the literature.

In this present technology, the term “stem cell” or “progenitor cell” will include (a) “stem cells and related cells obtained from or residing in the bone marrow” (including CD34+ cells); (b) “primitive or progenitor cells or related cells obtained from or residing in tissue compartments outside of the bone marrow” such as cells on the endothelial walls of blood vessels in general, and in particular mesenchymal stromal cells or cells obtained from the kidney (including “perivascular cells”) or cells obtained from any tissue. In other words, due to the different usages of these terms by different scientists, the term “stem cell” or “progenitor cell” in this disclosure would refer to cells that will eventually differentiate into the more mature and functional cells forming the tissues of an organ or a specific tissue, as found in healthy and functional tissues or organs, and studied by the pathologists or histologists.

Regarding the potential use of non-stem-cell methods to mitigate the harmful effects of renal dysfunction or damage, animal models of acute and chronic renal failures have been created, one of which is done by glycerol-induced rhabdomyolysis (GIR). The mechanism of GIR leading to acute kidney injury (AKI) has been proposed by Kim et al in the article published in Nephrology Dialysis Transplantation, Vol 25, Issue 5, May 2010, titled “N-acetylcysteine attenuates glycerol-induced acute kidney injury by regulating MAPKs and Bcl-2 family proteins.” Specifically, in their Introduction, the authors explained: Rhabdomyolysis is a syndrome involving the breakdown of skeletal muscle, which causes myoglobin and other intracellular proteins and electrolytes to leak into the circulation. It is often complicated by acute kidney injury (AKI), electrolyte imbalance and disseminated intravascular coagulation. About 10 to 50% of patients suffering from significant rhabdomyolysis develop some degree of AKI. Although the treatment has been much improved, the mortality rate may still be as high as 8%. The experimental model for rhabdomyolysis is easily acquired by injecting glycerol intramuscularly into rats or mice.”

The authors continued, “AKI by rhabdomyolysis has three pathogenic mechanisms: tubular obstruction, renal vasoconstriction and oxidative stress. Oxidative stress has been an important target in the prevention of myoglobin-induced renal injury. The administration of antioxidants has been shown to provide partial protection against myoglobinuric-induced AKI. N-acetylcysteine (NAC), one of these antioxidants, is a source of sulfhydryl and glutathione (GSH) groups in cells and, due to its interaction with reactive oxygen species, is a scavenger of free radicals. The protective effect of NAC with respect to renal injury has been proven in various models, such as cisplatin, ischemia-reperfusion injury and chronic kidney disease. However, there is little data for administering NAC in the rhabdomyolysis model, and the results are controversial.”

It should be pointed out that the administration of FPS to animals has resulted in favorable oxidative/reductive changes in some cell populations even long after only one dose of FPS. Specifically, FAS has an effect on the oxidative reactivity of cells, either directly or indirectly. The production of reactive oxygen species (ROS) is reduced in spleen cells harvested from irradiated animals even on day 51 after one treatment with one dose of FAS (Mao, 2014). However, there was no discussion or expectation in that work to show that FPS may be beneficial to remedy the harm of acute or chronic kidney failure. In other words, the disclosure here relating the administration of FPS to kidney problems is novel and non-obvious. It is within the realm of this present technology that FPS is expected to exert a positive effect on the repair of AKI via the beneficial effects of FPS as an inducer of anti-oxidants or its effect on the anti-oxidative pathway(s) inside cells.

Regarding the causes of cell damage, the article by Kim et al said, “There is conclusive evidence that renal tubular cells die by apoptosis as well as necrosis in experimental models. The c-Jun N-terminal kinase (JNK) and p38 pathways are activated in response to environmental stress, and this activation is frequently associated with the induction of apoptosis. Additionally, it is also known that the balance between cell survival and apoptosis is delicate, and the direction taken by the cell can be settled by activation of the extracellular signal-regulated kinase (ERK) and JNK/p38 kinase pathways. These mitogen-activated protein kinases (MAPKs) play an important role in determining the fate of renal tubular cells.” Therefore, it is within the expectations of this disclosure that the administration of FPS can exert its beneficial effects via mechanisms that (a) favor the healing of cells vs their apoptosis, (b) even in the scenario of necrosis (cell death) the mobilization of stem cells may accelerate the replacement of necrotic tissues by the rapid differentiation of stem cells that have arrived at the damaged sites inside or around the kidneys. In addition, this present technology envisions the possibility of FPS having a positive influence via (a) cell replacement mechanisms, or (b) paracrine effects (soluble molecules or small vesicles that work within short distances from their source); and both (a) and (b).

Kim also discussed other models causing AKI, “Rodent models of renal ischemia-reperfusion were associated with the activation of renal JNK and p38 but not ERK. Toxic renal injury, induced by mercuric chloride administration, was associated with two temporal peaks of renal ERK activation, whereas both renal ERK and JNK activation were increased in the glycerol model of myoglobinuric AKI. Thus, the evaluation of these pathways is considered essential for the therapeutic intervention in AKI. Moreover, it has not been studied how NAC affects the activation of MAPKs in the rhabdomyolysis.” We do not restrict our discussion in this disclosure about the exact mechanism of action of FPS in the healing of AKI because we expect that there would be multiple and possibly mutually-beneficial effects of more-than-one mechanisms from the administration of FPS. However, once the natural history of AKI from various causes have been established and the end-point (renal function recovery, from both acute and chronic models) has been established, we may then study the various pathways that have been induced by FPS, so that we can expand the benefits of FPS administration to other diseases.

Kim et al further hypothesized that:
(i) glycerol-induced renal dysfunction and histopathological changes are due to a decrease in the renal anti-oxidant reserves, and that these changes are correlated with activation of the MAPKs signaling leading to renal tubular apoptosis; and
(ii) NAC plays major renoprotective roles through controlling the signaling pathway which leads to the prevention of renal tubular apoptosis
   Their data supported their hypothesis. We expect the administration of FPS can lead to similar beneficial effects via similar mechanisms or a variety of other mechanisms.

Additional research work relating the effect of stem cells together with non-stem cell, chemical therapy was done by Mard et al, and published in “Inflammation, 2022 December; 45(6):2294-2308.” The title was “Gallic Acid Improves Therapeutic Effects of Mesenchymal Stem Cells Derived from Adipose Tissue in Acute Renal Injury Following Rhabdomyolysis Induced by Glycerol.” The author stated the importance of using gallic acid as an anti-oxidant: “The main limitation of mesenchymal stem cells (MSCs) therapy is reducing cell survival in response to oxidative stress products in injured organ areas. Gallic acid (GA) as a well-known antioxidant has been reported to confer potent free-radical scavenging and anti-inflammatory properties. Therefore, the aim of the current study was to assess the influence of MSCs and GA in acute renal injury following rhabdomyolysis induced by glycerol.” The authors concluded, “Our results revealed that co-treatment of AMCs plus GA into AKI rats decreased BUN and creatinine and ameliorated kidney injury parameters after 3 weeks. Improved oxidative stress markers such as decreased MDA and increased SOD and CAT were significant in the GA+AMCs group compared to the AMCs alone in AKI rats. Also, the histopathological appearances of AKI rats including renal tubule cavity expansion and renal tubular epithelial cell edema, and interstitial inflammation, were alleviated using GA+AMCs treatment compared to the control. The obtained results of the current study documented that antioxidants could make mesenchymal stem cells more resistant to the condition in which they are supposed to be transplanted and probably improve the efficacy of stem cell therapy in AKI patients.” It should be pointed out here that in this present technology, Yen is not introducing externally-sourced cells, thus avoiding the issues of tissue-incompatibility, potential contaminations, infections, difficulties with injection techniques and the associated costs
This present technology utilizes the patient's ability to produce/mobilize his or her own stem cells or progenitor cells which are totally compatible with the need of the injured kidney or other injured tissues or organs. Therefore, this disclosure is novel and non-obvious. It should also be pointed out that the infusion of FPS by itself has resulted in a favorably balance in the body toward the anti-oxidant state without the use of other agents. However, this present technology does not exclude the use of other chemical agents, including beneficial anti-oxidant agents which are safe and effective for use in combination with the administration of FPS.

The literature is full of examples of agents that can harm the kidney. In this disclosure we will reveal the impact of a chemotherapy drug called CisPlatin, which is administered to a wide variety of cancer patients despite its harm on the kidney. It can be appreciated that the present technology can have beneficial effects against the harmful impact to the kidneys by the use of chemotherapy agents such as, but not limited to, Cisplatin, Carboplatin (Paraplatin, Paraplatin AQ), Ifosfamide (Ifex), Methotrexate, Paclitaxel, Doxorubicin, Gemcitabine and the like.

The data will show that FPS can prophylactically reduce the harm done by this agent and may even treat the dysfunction or damage caused by this agent, after it has been administered to the patient. We believe that the beneficial effect of FPS against the harmful effect of CisPlatin is only one example of its efficacy, and that FPS will be found to be beneficial to reduce the harm of many other renal-harmful agents.

Although details of the present technology including dimensions, concentrations and other exact measures of the present technology have been disclosed here, it should be recognized that people skilled in the art can use different and various other dimensions and parameters, which will still be infringements of the nature and spirit of this present technology.

It has been discovered according to this present technology that the infusion intravenously of nanoparticles such as fibrinogen coated albumin spheres (FAS) into healthy subjects can result in an increase in certain populations inside the bone marrow, including stem cells which are CD34+ cells.

It has also been discovered according to this present technology that the infusion intravenously of nanoparticles such as FAS into injured subjects, including subjects suffering from rhabdomyolysis or any other muscular dysfunction (genetic or non-genetic causes, such as trauma) can result in an increase in certain populations inside the bone marrow, including stem cells which are CD34+.

It has also been discovered according to this present technology that the infusion intravenously of nanoparticles such as FAS into injured subjects, including subjects suffering from acute or chronic renal failure (or injury) of any cause can result in an increase in certain populations inside the bone marrow, including stem cells which are CD34+.

It has also been discovered according to this present technology that the infusion intravenously of nanoparticles such as FAS into injured subjects, including subjects suffering from muscle injuries and/or acute or chronic renal failure (or injury) of any cause can result in an increase in certain populations such as CD34+ stem cells in more than one tissue, including the previously-injured but recovering tissues including the kidney and or muscles.

It has also been discovered according to this present technology that the infusion intravenously of nanoparticles such as FAS into injured subjects, including subjects suffering from muscle injuries and/or acute or chronic renal failure (or injury) of any cause can result in an increase in cell populations positive for CD34 cell marker, or negative for CD34 cell markers, which are beneficial to the recovery of previously injured tissues, such as the kidney and or muscles.

It has also been discovered according to this present technology that the infusion intravenously of nanoparticles such as FAS into injured subjects, including subjects suffering from acute or chronic renal failure (or injury) of any cause can result in a favorable anti-oxidative status within the injured tissue leading to accelerated recovery of the injured tissue.

It has also been discovered according to this present technology that the infusion intravenously of nanoparticles such as FAS into injured subjects, including subjects suffering from acute or chronic renal failure (or injury) of any cause can result in an accelerated rate of improvement of the histological structures inside the kidney or the injured muscles, leading to improved function of the kidneys or muscles.

It has also been discovered according to this present technology that the infusion intravenously of nanoparticles such as FAS into injured subjects, including subjects suffering from acute or chronic renal failure (or injury) of any cause can result in the production of biological molecules or vesicles which have paracrine effects, leading to an accelerated rate of improvement of renal function.

It has also been discovered according to this present technology that the infusion intravenously of nanoparticles such as FAS into injured subjects, including subjects suffering from acute or chronic renal failure (or injury) of any cause can result in the production of biological molecules or vesicles which have paracrine effects, leading to an accelerated rate of improvement of the histological structures inside the kidney or the injured muscles, leading to improved function of the kidneys or muscles.

It has also been discovered according to this present technology that the infusion intravenously of nanoparticles such as FAS into injured subjects, including subjects suffering from acute or chronic renal failure (or injury) of any cause, but particularly from the administration of chemotherapy agents, can result in the production of stem cells, biological molecules or vesicles which have paracrine effects, leading to an accelerated rate of improvement of the histological structures inside the kidney, leading to improved function of the kidney.

It has also been discovered according to this present technology that the infusion intravenously of nanoparticles such as FAS into injured subjects, before or after their injury, including subjects suffering from acute or chronic renal failure (or injury) of any cause, but particularly from the administration of chemotherapy agents containing platinum, can result in the production of stem cells, biological molecules or vesicles which have paracrine effects, leading to an accelerated rate of improvement of the histological structures inside the kidney, leading to improved function of the kidney.

### EXPERIMENTS

### EXPERIMENT ONE: Beneficial Effects of FPS Administration after the Initiation of Rhabdomyolysis in a Rat Model

Purpose: to demonstrate the improvement in renal healing by the administration of a single dose of FPS soon after the administration of glycerol.

### Material and Methods

Various methods of inducing rhabdomyolysis have been published, including Nath KA et al, “Induction of heme oxygenase is a rapid, protective response in rhabdomyolysis in the rat” in J. Clin Invest, 1992, vol 90 (pages 267-270). Essentially, rhabdomyolysis-induced acute kidney injury was studied here in 6-week-old male C57Bl/6 mice by intramuscular injection on day 0 with hypertonic glycerol (8 ml/kg body weight of a 50% glycerol solution; Sigma-Aldrich) into the inferior hind limbs, as has been described previously also by Angelotti ML, Ronconi E, Ballerini L et al “Characterization of renal progenitors committed toward tubular lineage and their regenerative potential in renal tubular injury” published in Stem Cells 2012; 30:1714-1725.

This model induces actually two injuries: (a) that of renal failure due to the obstructive effects of the disintegrating muscle molecules, and also (b) muscle disintegration in the leg. This is, therefore, a double injury model. We therefore study the beneficial effect of FPS in both the renal function as well as wound recovery in the leg. The fact that there is more than one site or modality of injury fits into the realm of “polytrauma” as discussed in the application “Protein Nanoparticles to Treat Harm from Multiple Trauma.” USPTO Publication number 20230241182, published on Aug. 3, 2023.

FPS was manufactured as described in Yen 2016 (“Mass Production of Ready-to-Use Suspensions of Fibrinogen-coated Albumin Spheres for the Treatment of Thrombocytopenic Patients”: US Publication number 20160354481, published on Dec. 8, 2016 by USPTO). Essentially, human serum albumin (HSA) solutions (5.5% diluted from 25% excipient-grade of HSA with water) was added ethanol solutions to result in a turbid suspension of “blank albumin spheres.” Subsequently, the spheres are coated with human fibrinogen to form a suspension of Fibrinogen-coated Albumin Spheres (FAS.) After adjustment of osmolarity and the addition of other excipients, the drug product is filled into sterile bottles, which are then treated with a terminal step of pasteurization at 60 deg C. for 10 hours. The bottled, sterile product (100 mL per bottle, containing 800 mg of FAS per bottle) is called Fibrinoplate-S (FPS) which is used in these experiments.

In most prior-art studies, the rescue drug or chemical is administered either prior to glycerol injection (e.g. Kim et al) or within 24 hour post-glycerol injection (e.g. Korrapati et al, in “Recovery from Glycerol-Induced Acute Kidney Injury Is Accelerated by Suramin” in J Pharmacol Exp. Ther. 2012 April; 341 (1): 126-136.) Here we administered the FPS intravenously via the tail vein of the rats at one-hour post-glycerol injection (i.M.) so that there will be adequate time for the mobilization of stem cells from the bone marrow or from a perivascular compartment (in the kidney or elsewhere.) The timing is designed to allow the “rescue cells” (whatever the cells may be, or their effects in producing a paracrine effect) to arrive in time at the damaged locations (either the kidney or the leg, or both) to start the beneficial work of rescue.

The public literature on renal failure studies has indicated that the rat species is typically better than the mouse species, in that for some unknown reasons, the same injurious agent that will cause renal failures in rats may not do so in mice. Therefore, we will use a rat model for this experiment.

The administration of FPS for other conditions, such as irradiation-induced skin injury has been shown to be effective whether the FPS was given (intravenously) (a) before the irradiation event, which is called the “prophylactic approach”, or (b) immediately after the irradiation event, but before the appearance of a skin lesion or injury, which is called “mitigation approach”, or (c) soon after the appearance of the skin lesion, which is called “therapeutic approach.” There is no exclusion of one approach versus another. We expect that in these studies on AKI, the beneficial effects may be seen from any of the three above approaches, although there will be differences among these methods in terms of ease of application, or the speed of healing or the degree of healing. The same is true with respect to massive muscle injuries, which can be caused by chemicals, trauma, blunt injuries, burns: both in war situations as well as non-combat situations.

We followed the recovery from AKI for 10 days (in contrast to only 5 days as in Korrapati et al). There are 4 groups (n=6). Group 1 is control injected with 0.5 mL saline per kg weight of the animal, sacrificed on day 5. Group 2 is treated with FPS 4 mg per kg (equal to 0.5 mL per kg) sacrificed on day 5. Group 3 is control injected with 0.5 mL saline per kg weight of the animal, sacrificed on day 10. Group 4 is treated with FPS 4 mg per kg (equal to 0.5 mL per kg) sacrificed on day 10. Blood chemistry including renal function tests (BUN, Cr, Cr-clearance) were sampled on Day 0, and at 24, 48, 72 and 120 hour post injury. Kidney histology and leg muscle pathology were studied for biopsies obtained after sacrifice from all four groups.

### Results

For reference, FIG. 1 of Korrapati et al (“Recovery from Glycerol-induced Acute Kidney Injury is Accelerated by Suramin” J Pharmacol Exp Ther 2012 April; 341(1):126-136) is provided with this description. The data shown in Panel A is for serum creatinine, and the data shown in Panel B is for urine creatinine.

Referring to Panel A: it can be seen that the serum creatinine in the grey bars (glycerol group) was consistently higher in the injured group from 24 hr to 120 hr (compared to untreated, i.e. non-glycerol treated group.) The data also show that the group with suramin treatment (the black bars) after glycerol injury is improved with respect to serum creatinine level compared to the glycerol-without-suramin group.

We report here that the administration of FPS at 4 mg per kg wt of the animal injected at one hour post-glycerol-administration resulted in beneficial effects on serum creatinine similar to those seen in Panel A in FIG. 1.

Histological (pathological) studies on the biopsies on both the kidneys and the leg muscles also show improvements in the FPS-treated group compared to the control saline-group, in both samples taken on day 5 and on day 10.

Further studies are being conducted to stain for CD34+ cells as well as for other cell markers related to recovery of muscles, blood vessels, and other kidney morphologies and cell markers disclosed by Korrapati et al.

### Comments

The intravenous administration of FPS at a dose of 4 mg per kg is effective in mitigating the harm caused by glycerol administered at the leg. Preliminary studies in the concentration of CD34+ cells in the kidney as well as the muscles in the leg suggest that stem cells from the bone marrow or other progenitor sites were involved in the mobilization of rescue cells to result in recovery. It is also possible that the effect of FPS includes the production of cells which have a paracrine effect on the speedy recovery of kidney function. Although the model used here is acute kidney failure, we expect that FPS is similarly effective in the treatment of chronic kidney failure. Although glycerol was used as the injurious agent here, it is expected that a similar beneficial effect of FPS on the recovery of renal dysfunctions or damage caused by a wide variety of injuries including, but not limited to, diabetic renal failures, Autosomal Dominant Polycystic Kidney Disease (ADPKD), Minimal Change Disease (MCD), and other kidney dysfunctions widely publicized in the public domain.

### EXPERIMENT TWO: Beneficial Effects of FPS Administration in a Rat Model of Renal Injury Caused by Anti-Cancer Chemotherapeutic Agents

### Introduction

The previous Experiment One uses rhabdomyolysis as an agent of harm, causing renal tubular obstruction from the clogging effects of the disintegrating muscle fibers happening elsewhere in the body (i.e. in the leg muscles.) Experiment Two uses a different model that is not primarily related to the obstruction of the tubular structures of the kidney, i.e. a use of the specific damaging effect of a chemotherapeutic agent to cause structural damage in the kidney. Many such chemotherapeutic agents are known; Experiment Two uses cisplatinum (brand name CisPlatin) here because it is widely used to treat a number of cancers, including ovarian, testicular, cervical, head and neck, and lung cancers. The mechanism of damage is published: “Mechanisms of Cisplatin Nephrotoxicity” in Toxins (Basel): 2010 November; 2(11): 2490-2518, authored by Miller et al.

Regarding Biomarkers for kidney injury, one of the most sensitive biomarkers is Osteopontin (OPN) which is located mainly in the kidney, bone and a few other organs. See: “Osteopontin as a Biomarker in Chronic Kidney Disease” by Sinha et. Al., in Biomedicines, 2023 May; 11(5):1356. The authors stated that “Among all tissues, kidneys have the greatest OPN content. In normal kidneys, OPN is mainly expressed in the loop of Henle and the distal nephron; but following kidney damage, its expression is upregulated in all tubular segments and in the glomeruli by as much as 18-fold”-see “Expression roles, receptors, and regulation of osteopontin in the kidney” by Xie et al, in Kidney Int. 2001; 60:1645-1657.

### Materials and Methods

Fibrinoplate-S (FPS) used in this experiment is the same Lot as described in Experiment One, containing 8 mg of FAS per mL of suspension. CisPlatin (CisP) was purchased from a commercial supplier, administered to the rats as indicated in Table 1 below on Day zero, at 7 mg per kg wt of the animal, intraperitoneally (IP). The rats were not given extra fluid for additional hydration which is a standard prophylactic treatment in human patients for the renal toxicity caused by CisP.

There are 5 groups of rats (n=4), as described in Table 1 below.

TABLE 1

| | | | Draw Blood on Day | Test Urine from |
|---|---|---|---|---|
| | | | 3, 5, 10 for renal | Day 5, 10 for |
| | CisP, | FPS, | function tests | osteopontin |
| Group | 7 mg/kg | 1 mL/kg | and electrolytes | concentration |
| 1 | none | none | Yes | Yes |
| 2 | On Day 0 | Day −3 | Yes | Yes |
| 3 | On Day 0 | Day 0, | Yes | Yes |
| | | after CisP | | |
| 4 | On Day 0 | Day 3 | Yes | Yes |
| 5 | On Day 0 | Saline on | Yes | Yes |
| | | Day 3, No | | |
| | | FPS | | |

### Results

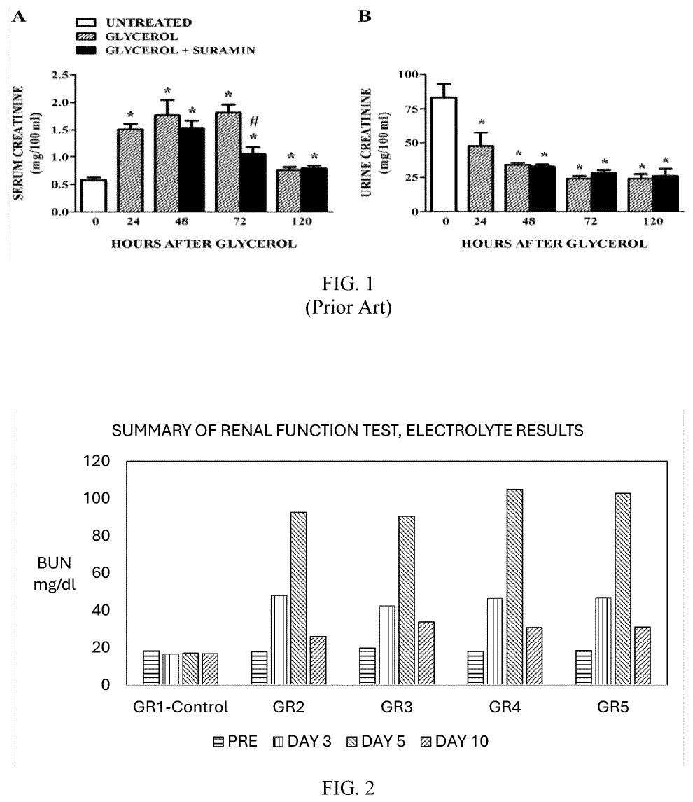

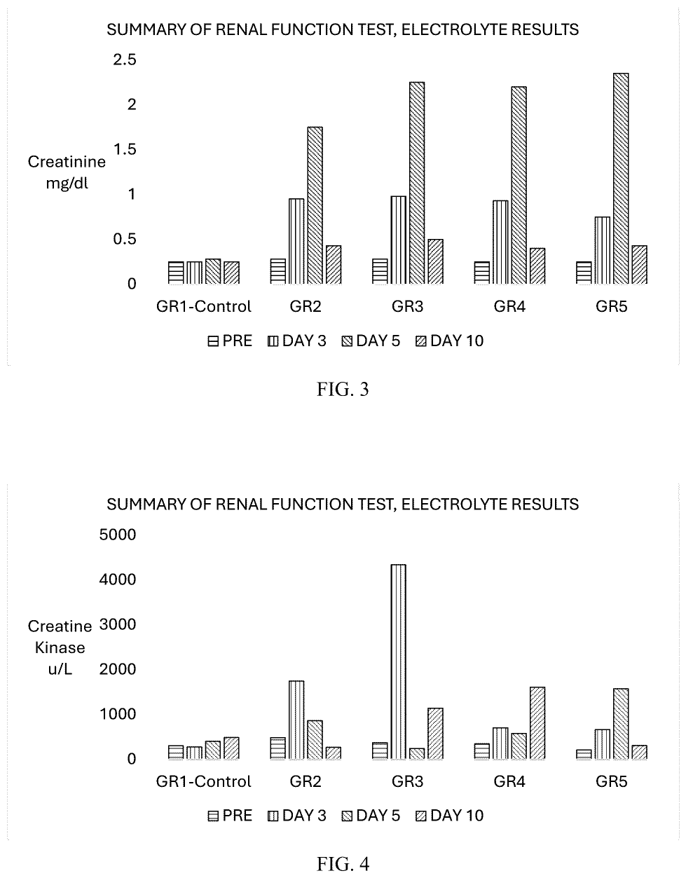

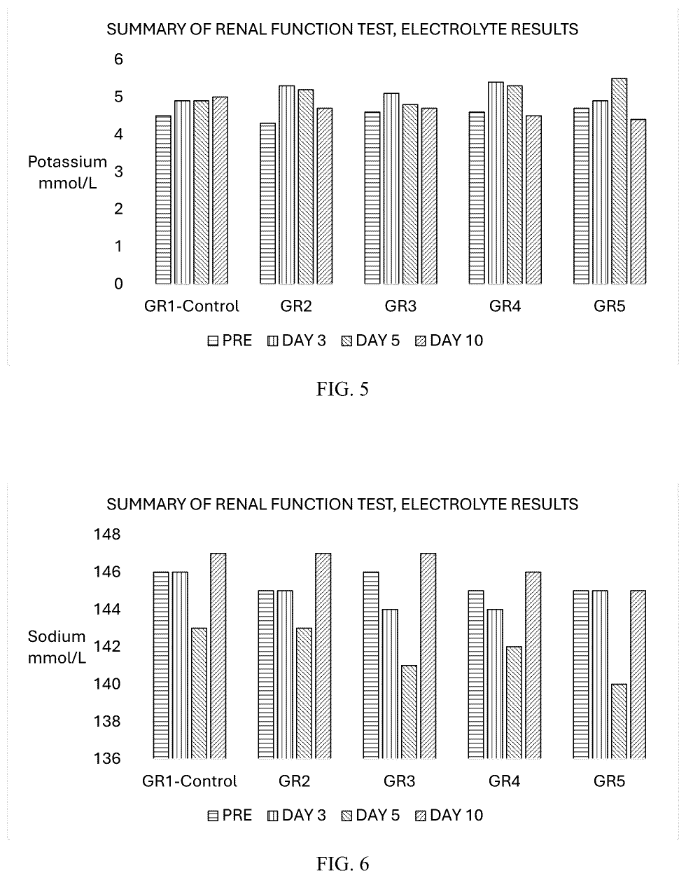

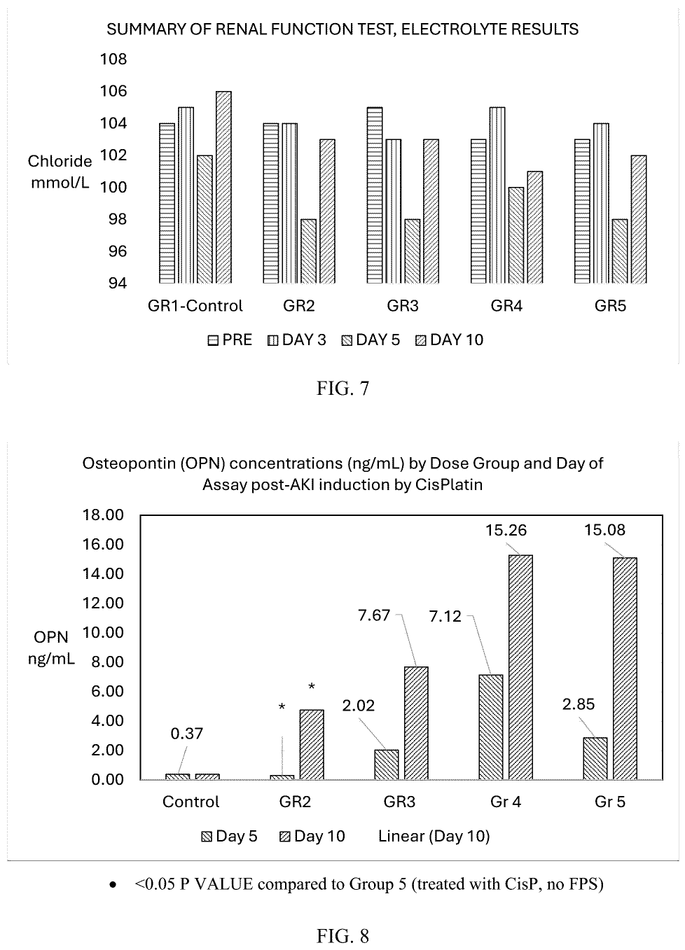

The following Tables 2-6 show the results of the assays on urinary osteopontin, various serum renal function tests (BUN, Creatinine, Creatine Kinase) and the serum electrolytes (Sodium, Potassium, Chloride) on various days pre- or post-CisP administration. The term “PRE” refers to samples being taken “pre-CisPlatin” whether it is taken on the same day before CisPlatin is administered, or on the day before. FIGS. 2-7 graphically illustrate the results shown in Table 5, while FIG. 8 graphically illustrates the mean urine OPN concentration in each group.

TABLE 2
URINE OSTEOPONTIN CONCENTRATION

| OSTEOPONTIN (ng/mL) | DAY 5 | DAY 10 |
|---|---|---|
| GROUP 1 | 0.37 | 0.37 |
| GROUP 2 | 0.29 | 4.74 |
| GROUP 3 | 2.02 | 7.67 |
| GROUP 4 | 7.12 | 15.26 |
| GROUP 5 | 2.85 | 15.08 |

TABLE 3
OPN CONCENTRATION IN EACH GROUP
COMPARED TO GROUP 1: P VALUES

| OSTEOPONTIN (ng/mL) | DAY 5 | DAY 10 |
|---|---|---|
| GROUP 2 | 0.402 | 0.092 |
| GROUP 3 | 0.230 | 0.124 |
| GROUP 4 | 0.042 | 0.021 |
| GROUP 5 | 0.052 | 0.001 |

TABLE 4
OPN CONCENTRATION IN GROUP 2, 3, 4 COMPARED
TO GROUP 5: P VALUES (For comparison of
Group 1 with Group 5, see Table 3)

| OSTEOPONTIN (ng/mL) | DAY 5 | DAY 10 |
|---|---|---|
| GROUP 2 | 0.048 | 0.022 |
| GROUP 3 | 0.692 | 0.220 |
| GROUP 4 | 0.184 | 0.974 |

TABLE 5
SUMMARY OF RENAL FUNCTION
TEST, ELECTROLYTE RESULTS

| | PRE | DAY 3 | DAY 5 | DAY 10 |
|---|---|---|---|---|
| BUN (mg/dL) | | | | |
| GROUP 1 | 18.3 | 16.5 | 17.0 | 16.8 |
| GROUP 2 | 17.8 | 47.8 | 92.5 | 26.0 |
| GROUP 3 | 19.8 | 42.3 | 90.5 | 33.8 |
| GROUP 4 | 18.0 | 46.3 | 104.8 | 30.8 |
| GROUP 5 | 18.5 | 46.5 | 102.8 | 31.0 |
| CREATININE (mg/dL) | | | | |
| GROUP 1 | 0.25 | 0.25 | 0.28 | 0.25 |
| GROUP 2 | 0.28 | 0.95 | 1.75 | 0.43 |
| GROUP 3 | 0.28 | 0.98 | 2.25 | 0.50 |
| GROUP 4 | 0.25 | 0.93 | 2.20 | 0.40 |
| GROUP 5 | 0.25 | 0.75 | 2.35 | 0.43 |
| CREATINE KINASE (u/L) | | | | |
| GROUP 1 | 301.5 | 274.3 | 397.3 | 483.3 |
| GROUP 2 | 480.5 | 1740.0 | 857.8 | 264.3 |
| GROUP 3 | 367.3 | 4332.8 | 236.5 | 1136.3 |
| GROUP 4 | 342.5 | 698.0 | 575.0 | 1603.5 |
| GROUP 5 | 207.0 | 660.3 | 1571.0 | 304.7 |
| POTASSIUM (mmol/L) | | | | |
| GROUP 1 | 4.5 | 4.9 | 4.9 | 5.0 |
| GROUP 2 | 4.3 | 5.3 | 5.2 | 4.7 |
| GROUP 3 | 4.6 | 5.1 | 4.8 | 4.7 |
| GROUP 4 | 4.6 | 5.4 | 5.3 | 4.5 |
| GROUP 5 | 4.7 | 4.9 | 5.5 | 4.4 |
| SODIUM (mmol/L) | | | | |
| GROUP 1 | 146 | 146 | 143 | 147 |
| GROUP 2 | 145 | 145 | 143 | 147 |
| GROUP 3 | 146 | 144 | 141 | 147 |
| GROUP 4 | 145 | 144 | 142 | 146 |
| GROUP 5 | 145 | 145 | 140 | 145 |
| CHLORIDE (mmol/L) | | | | |
| GROUP 1 | 104 | 105 | 102 | 106 |
| GROUP 2 | 104 | 104 | 98 | 103 |
| GROUP 3 | 105 | 103 | 98 | 103 |
| GROUP 4 | 103 | 105 | 100 | 101 |
| GROUP 5 | 103 | 104 | 98 | 102 |

TABLE 6
COMPARISON OF VARIOUS GROUPS IN TABLE
FIVE (5) THAT ARE STATISTICALLY SIGNIFICALLY
DIFFERENT FROM GROUP 1: P VALUES < 0.05
(Comparisons with P Values > 0.05 are left
blank in this table and not listed)

| | | | |
|---|---|---|---|
| PRE | DAY 3 | DAY 5 | DAY 10 |

| | | | |
|---|---|---|---|
| BUN (mg/dL) | | | |
| GROUP 2 | 0.0001 | 0.012 | 0.02 |
| GROUP 3 | 0.014 | 0.012 | 0.004 |
| GROUP 4 | 0.0036 | 0.0005 | 0.04 |
| GROUP 5 | 0.0012 | 0.00002 | 0.0204 |
| CREATININE (mg/dL) | | | |
| GROUP 2 | 0.000001 | 0.013 | 0.0038 |
| GROUP 3 | 0.015 | 0.014 | 0.008 |
| GROUP 4 | 0.003 | 0.004 | 0.012 |
| GROUP 5 | 0.008 | 0.0019 | 0.022 |
| CREATINE KINASE (u/L) | | | |
| GROUP 2 | | | |
| GROUP 3 | | | |
| GROUP 4 | 0.015 | | |
| GROUP 5 | | | |
| POTASSIUM (mmol/L) | | | |
| GROUP 2 | | | |
| GROUP 3 | | | |
| GROUP 4 | | 0.022 | |
| GROUP 5 | | | |
| SODIUM (mmol/L) | | | |
| GROUP 2 | | | |
| GROUP 3 | 0.019 | 0.042 | |
| GROUP 4 | | 0.011 | |
| GROUP 5 | 0.040 | 0.020 | |
| CHLORIDE (mmol/L) | | | |
| GROUP 2 | | 0.037 | |
| GROUP 3 | | 0.047 | |
| GROUP 4 | | 0.017 | 0.01 |
| GROUP 5 | | | |

### Discussion

This experiment aimed at showing (a) the damaging effect of a chemotherapeutic drug called CisPlatin; (b) the mitigating effect of FPS administration. Three different methods of detection were used: namely that of osteopontin, renal function tests, and electrolyte disturbances. It should be noted that renal function tests are tests conducted on samples of blood, designed to show renal dysfunction, which may or may not be the result of actual damage to the kidney. For example, serum BUN can be increased due to physiological changes such as dehydration, which can be reversed without any damage to the kidney or kidney cells. On the other hand, a stressful condition on the kidney cells (such as by some chemotherapeutic agents) may also lead to abnormal renal function test before the kidney cells actually die and become integrated. Therefore, the peak of dysfunction (indicated by renal function test) can be on a day different from the period where the kidney cells cannot withstand the stress anymore and start to die. When the kidney cells actually die, the biomarker osteopontin will spill into the urine. Thus, the peak of osteopontin in the urine, which reveals actual kidney damage, will be on a day different (typically delayed compared to the results of renal function tests) from the day of peak-dysfunction indicated by BUN measurements.

The FPS was administered uniformly at 1 mL per kg weight of the animal, but at 3 different time-points: on Day-3, Day 0, Day 3. The hypothesis is mainly that FPS can mobilize stem cells toward a wound (in this case the kidney damage caused by CisPlatin). Therefore, it is expected FPS administered on Day-3 to be most effective, because the stem cells have already been mobilized before CisPlatin was administered on Day 0. FIG. 8 showed that FPS administered on Day-3 was most effective, more so than on Day 0 and practically not effective when administered on Day3 when the damage is already done, in terms of OPN concentrations released into the urine.

Public literature has an abundance of information on the numerous variations of OPN: e.g. the “full length” OPN can be cleaved by various proteases including thrombin, matrix metalloproteinase (MMP)-3, MMP-7, cathepsin-D and plasmin, producing N-terminal OPN (ntOPN) and other variations or derivatives of the full-length OPN. See “Osteopontin as a Biomarker in Chronic Kidney Disease” by Sinha et al in Biomedicines. 2023, May; 11(5): 1356. In this disclosure, the use of the term “OPN” includes all of the varieties or derivatives of OPN that have been studied or yet to be discovered. The same authors also said “Targeting OPN may be a potential treatment strategy. Several studies show that inhibition of OPN expression or activity can attenuate kidney injury and improve kidney function.” Therefore, it is not ruled out the possibility that the beneficial effects of FPS administration may not be limited to the mechanism of stem cell mobilization but can include the inhibition of the expression or release of OPN, leading to less damage to the kidney caused by CisPlatin or any other renal-damaging agents.

The best way to study how FPS can mitigate renal damage or dysfunction may involve the histological/pathological study of the kidney at various stages of damage: to show that the histological damage is less severe or on the way to recovery in the FPS-group, due to repair of the tubules or the blood vessels or whatever the site of the damage may be. To provide evidence of the mechanism of action: staining for stem cells (CD34+) and other more differentiated cells in the kidney should also show that they are more abundant in the FPS-group than in the control group.

Regarding the use of Osteopontin (OPN) as a marker for kidney damage and the mitigation of harm (or acceleration of healing) by FPS, the data show that OPN is a sensitive biomarker which proved that there is definitely renal damage by the administration of CisPlatin (see Table 2: Group 5 compared to Group 1). The OPN urine concentration is raised from 0.37 ng/mL in the control untreated (Group 1) rats to 2.85 and 15.08 ng/mL (Group 5) on Day 5 and Day 10, respectively. This is a 40× increase in OPN concentration.

Table 3 showed the P values by comparison of the various Groups to Group 1 (by T-test). Due to the small sample size and the sensitivity of the OPN test, a comparison of the OPN values in Group 5 with that of Group 1 on Day 5 did not show a statistically significant difference by a small margin (P value being 0.052); even though on Day 10 there is a major difference (P=0.001). Comparison of Group 4 (with FPS administered on Day 3) with Group 1 showed a statistically significant difference on both Day 5 and Day 10. However, when Group 2, 3 are compared to Group 1, there is no statistically significant difference between them (P>0.05)—meaning that FPS may have a protective effect even if administered on Day 0 (Group 3), although the average OPN concentration in Group 3 was raised on both Day 5 and Day 10 (to 2.02 and 7.67 ng/mL, respectively) when compared to the untreated control (Group 1) on these days.

Table 4 showed the P values of the comparison of the various groups with Group 5. These comparisons are aimed at showing whether FPS administration on a particular day will show significant protection against the harm caused by CisPlatin (Group 5). The data showed that only Group 2 is significantly different from Group 5) having P values of 0.048 and 0.022 on Day 5 and Day 10, respectively.

FIG. 8 showed the average value of OPN concentration on Day 5 and 10, in Group 1, 2, 3, 4 and 5. It can be readily seen that the OPN level in Group 2 is very different from Group 5, and it is almost identical to those of Group 1. It should be noted that Day 5 refers to the fifth day after CisPlatin injection, which is 8 days post-FPS administration. Still the protective effect of this prophylactic dose of FPS is seen. However, the protective effect is less obvious in Group 3 and even less so in Group 4.

Regarding the OPN values on Day 10, it can be observed that Group 2 is significantly better (lower) than Group 5; and Group 2 is better protected than Group 3 and Group 4.

A comparison of the day when renal dysfunction is the highest (e.g. FIG. 2, using BUN) vs the day where renal damage is the worst reveals that renal dysfunction is the worst on day 5, e.g. in Group 2 (the prophylactic treatment) whereas the worst day of renal damage (by OPN level in the urine, in FIG. 8, using also Group 2) is day 10. Although only two time-points (day 5 and day 10) were used in the measurement of OPN level in the urine, there is no question that day 5 is much better than day 10 in terms of the absence of renal damage, because the OPN level on day 5 (e.g. in Group 2) is essentially the same as the control (Group 1) on day 5 (no damage.) This is not surprising because renal dysfunction can be caused by “stress” on the kidney before its cells actually die. Only when the kidney cells start to disintegrate (after day 5), then OPN is spilled into the urine, to be revealed in the increased urine OPN level on day 10.

The data suggested that stem cell mobilization is likely to be the major mechanism of action. It has been previously reported in “Protein Nanospheres to Treat Harm from Multiple Trauma” (U.S. patent application Ser. No. 18/295,829, received by the USPTO on Apr. 4, 2023) in Experiment Two, that “The data showed that the FPS induced a significant increase in “CD34+ve, lineage cocktail −ve cells” in the bone marrow at days 1 and 5 (FIG. 8, P<0.05 for each time point). Approximately 2% of bone marrow cells were positive for CD34 and negative for “lineage cocktail” in the bone marrow of control animals. The maximum increase of around 50% was seen on Day 1. By Days 10-14, the percentage of this subset was reduced and was not significantly different from that in the controls (FIGS. 9 and 10 of U.S. patent application Ser. No. 18/295,829).”

In this experiment, CisPlatin was administered on Day 0 while FPS in Group 2 was administered on Day-3. Therefore, there are plenty of stem cells ready to move to the kidney as soon as the harm is detected there. In contrast, administration of FPS on Day 0 (Group 3) would be less effect than in Group 2 because the harm is exerted probably before the stem cells in the bone marrow can be mobilized. Consistent with this scenario, mobilization of stem cells after the harm has been going on for days (as in Group 4) would negate the effect of stem cell mobilization because the harm has been done, as reflected by the massive release of OPN into the urine in Group 4.

The data here suggest that the effect of FPS is mainly prophylactic, i.e. that FPS should be given before the administration of CisPlatin. However, it is still possible that FPS can have a “mitigating effect” or even a “therapeutic effect” over the entire cycle of CisPlatin harm, i.e. FPS can be beneficial even when given after a definite harm has been produced. However, such “late” effects of FPS will require the study of biopsies from various groups, to compare the histological differences between groups having “CisPlatin plus FPS-at-various-times” vs the Group inflicted with CisPlatin alone. The histological data may show that FPS administration can reduce the duration of harm or the severity of harm, or both, from a variety of renal toxic substances. The abundance of CD34+ stem cells in the biopsy of the kidneys from FPS-treated groups compared to the control group will support the mechanism of action being the mobilization of stem cells toward the healing of the kidney regardless of when FPS is administered.

The prophylactic benefit of FPS in these experiments is consistent with the mobilization of stem cells. Apparently, the administration of CisPlatin after the mobilization of the stem cells does not harm the stem cells. This is in contrast to the results observed regarding the effect of FPS over irradiation harms. Yen has disclosed that the beneficial effect of FPS for radiation harm when FPS is administered before the irradiation event is much less than if FPS is administered after the irradiation event. This is because irradiation will kill stem cells; therefore, it is better to mobilize the stem cells only after the irradiation event has passed. See Yen disclosure in US non-provisional number 11,260,110 “Nanoparticles for the Therapeutic Treatment of Radiation-induced Skin Ulcers” issued Mar. 1, 2022. In both event, however, stem cell mobilization is still the best explanation for the observed effects.

The data here suggest strongly that a second or even a third dose of FPS would benefit the patient. If in Group 2, the FPS is given on Day-3 and the protective effect is still observed on Day 5, but not so much on Day 10, then a reasonable interval of a multiple-dose FPS administration would be 5 or fewer days. Therefore, we expect this regiment to work well: a multiple-dose regiment of FPS on Day-3, Day 2 (for two doses) and Day-3, Day 2, and Day 7. It is obvious that a variety of FPS administration regiments may be tried to find out the most optimal usage of FPS administration.

We have not studied the whole cycle of harm caused by CisPlatin and if any spontaneous healing of the kidney can occur. It is clear from FIG. 8 that OPN spillage into the urine occurred as soon as 5 days post-CisPlatin, and worse on Day 10. If we increase the duration of study, we may find that OPN levels may rise even more than 15 ng/mL later or it may return to normal eventually (or not). Knowing the duration of harm to the kidney from one dose of CisPlatin will help us determine how many doses of FPS will be needed to offer complete coverage and mitigation of the harm done by one dose of CisPlatin; or even multiple doses of CisPlatin, which are sometimes needed to treat certain tumors in human patients.

Regarding the use of the Renal Function Tests, the data showed that BUN, Creatinine and Creatine Kinase are “early” indicators of kidney dysfunction but not necessarily helpful indicators of kidney damage or its repair. It should be noted that dysfunction can occur with or without structural damage. Since structural damage is typically harder to repair than dysfunction, because new cells corresponding to the damaged cells will need to come in and start working, scientists will typically agree that evidence of structural repair will be more important than evidence of the recovery from dysfunction.

On the comparative importance of dysfunction vs damage, one may use the illustration of a water pipe: if it leaks, it shows dysfunction, because structurally it still allows water to pass through. However, if it breaks, it is a “structural damage” typically resulting in a failure to serve the purpose of being a conduit for the water to pass through. Although it can be argued about the “extent” to which a pipe needs to show “structural damage” before it is considered broken, the two descriptions (“dysfunction” vs “damage”) both indicate suboptimal operation of the system, thus needing “repair” or “mitigation.” The reason we include the Renal Function Tests in this experiment is because these are “standard blood tests” while osteopontin is not a routine test for kidney intactness. Table 5 showed that as early as Day 3 after CisPlatin administration, BUN, Creatinine, and Creatin Kinase are all elevated, even in Group 2 where OPN level on Day 5 is similar to the control (Group 1.) In fact, the Creatine Kinase in Group 2 and 3 on Day 3 is higher (1740 and 4332 u/L, respectively) than that of Group 5 (660 u/L). It should be noted that BUN, Creatinine and Creatine Kinase levels are all subject to physiological changes and thus are not exclusively indicative of kidney damage, until the values are highly abnormal. For example, a high BUN level can be due to dehydration, urinary tract obstruction, congestive heart failure, or recent heart attack. Therefore, to study the beneficial effects of FPS on kidney restoration, one must include the study of useful markers on the structural integrity (or lack thereof) of the kidney, such as OPN.

Again, whether it is dysfunction or damage, it would be helpful to have data over the “life cycle” of one dose (or a regiment of multiple doses) of CisPlatin so that one can track the onset and the duration of harm and if possible when the kidney can return to normal spontaneously. Then, the effect of a single dose of FPS (or multiple doses) to mitigate the harm from CisPlatin can be studied, including the best timing of the FPS dose(s).

Table 6 showed the P values obtained by T-tests when various groups are compared to Group 1. The data showed that BUN and Creatinine levels have become abnormal as soon as Day 3 (and possibly before Day 3.) The data also show that Creatine Kinase as a marker is not very useful: it is statistically significant from that of Group 1 on only one occasion, that of Day 3 in Group 4, even though CisPlatin is obviously causing much harm (mainly dysfunction) to the kidney by then.

Regarding the use of serum electrolytes as markers for kidney dysfunction or damage, the data showed that Potassium, Sodium and Chloride concentrations are not useful in that regard. This is probably because they are essential for life and need to be within a narrow range for life to function, i.e. that these values will not vary substantially from the “normal range” until the patient is probably near death. These markers are far less useful than can be obtained by the study of urine OPN levels (and histological studies.)

We did not study the effect of FPS on the cytokines here in the context of renal failure. However, previous studies have shown the effects of FPS in the suppression of pro-inflammatory cytokines and the promotion of “good” cytokines. See: Radioprotective Efficacy of Fibrinogen-coated Albumin Spheres (FAS) Against Radiation-induced Skin Damage, by Mao et al, published in Rad Res. 2019, Sep. 13. We believe that in the environment of kidney failure FPS is beneficial in that it can suppress the pro-inflammatory cytokines and promote useful cytokines helpful to the recovery from renal failure.

Although we use CisPlatin as a nephron-toxic agent here, the literature has a wide variety of agents that can be harmful to the kidney. As shown in Table 7, which is reproduction of Table 1 as published in World J Clin Oncol. 2020 Apr. 24; 11(4): 190-204, titled “Nephrotoxicity in cancer treatment: An overview” authored by Maria Luísa Cordeiro Santos, is reproduced herewithbelow. We believe that FPS can be beneficial to reduce the harm caused by these agents, or accelerate the healing of the harm done.

TABLE 7
Relationship between anticancer drug class, its nephrotoxic
effects and mechanism of action of its lesion

| Class | Drugs | Nephrotoxicity | Mechanism of action |
|---|---|---|---|
| Alkylating agents | Bendamustine; | AKI; Hemorrhagic | Damage to proximal |
| | Cyclophosphamide; | cystitis; Inflammatory | and distal tubular |
| | Ifosfamide; | lesion; SIADH; | structures by action of |
| | Melfalano; | Hyponatremia; | metabolites and |
| | Nitrosureasnts | Fanconi's syndrome; | increased cellular |
| | | Interstitial nephritis; | oxidative stress |
| | | Diabetes | |
| Antimetabolites | Chlopharabine; | AKI; Decreased GFR; | Decreased GFR due to |
| | Methotrexate; | Interstitial edema; | vasoconstrictor action |
| | Pemetrexed; | Tubular acidosis; | on afferent renal |
| | Gemcitabine; | Diabetes insipidus; | arteries; Crystal |
| | Pentostatin | Microangiopathic | precipitation in tubules |
| | | hemolytic anemia; | and induction of |
| | | SIADH; | tubular injury |
| | | Hyponatremia | |
| Anti-microtubular | Paclitaxis; | SIADH | Inhibits synthesis of |
| agents | Vincristine; | | genetic material or |
| | Vinblastine; | | causes irreparable |
| | Vinorelbine | | DNA damage |
| Antitumor | Daunorubicin; | Nephrotic syndrome; | Epithelial lesions |
| antibiotics | Doxorubicin; | Focal segmental | (podocytes) |
| | Mitomycin | glomerular sclerosis; | |
| | | TMA; AKI; | |
| | | Hemolytic uremic | |
| | | syndrome | |
| Platinum agents | Cisplatin; | AKI; Anemia; | Drug accumulation in |
| | Carboplatin; | Hypomagnesemia; | the proximal renal |
| | Ocaliplatin | Proximal tubular | tubules |
| | | dysfunction; TMA | |
| Cytotoxic agents | Arsenic trioxide; | Tubulointerstitial | Increased exposure can |
| | Etoposide; | disease; | be toxic; Higher levels |
| | Irinotecan; | Rhabdomyolysis; AKI | of hematologic toxicity |
| | Topotecan | | |
| Immunomodulatory | Thalidomide; | Hypercalcemia; | Unclear, depending on |
| drugs | Lenalidomide; | Decreased GFR; | the drug, may be |
| | Pomalidomide | Nephrolithiasis | related to its type of |
| | | | metabolism |
| Proteasome | Bortezomib; | TMA; Acute | Prerenal causes (e.g., |
| inhibitors | Carfilzomib; | interstitial nephritis; | hypovolemia); Tumor |
| | Ixazomib | AKI | lysis-like syndrome |
| EGFR pathway | Cetuximab; | Electrolyte | Inhibition of EGFR |
| inhibitors | Panitumumab; | disturbance; AKI; | signaling at the distal |
| | Afatinib; Erlotinib; | Diffuse proliferative | convoluted tubule, |
| | Gefitinib | glomerulonephritis; | which regulates |
| | | Nephrotic syndrome | transepithelial |
| | | | magnesium transport; |
| | | | AKI mechanism is |
| | | | unclear, however, |
| | | | EGFR plays a role in |
| | | | the maintenance of |
| | | | tubular integrity |
| HER-2 inhibitors | Trastuzumab; Ado- | Proteinuria; AKI; | Unclear |
| | trastuzumab | Decreased GFR; | |
| | emtansine; | Electrolyte | |
| | Pertuzumab | disturbance; | |
| | | Hypertension | |
| BCL-2 inhibitors | Venetoclax; | AKI | Tumor lysis syndrome |
| | Obituzumab; | | |
| | Ofatumumab | | |
| ALK inhibitors | Crizotinib; Alectinib; | Decreased GFR; | Unclear |
| | Brigatinib | Development of | |
| | | complex renal cysts; | |
| | | Electrolyte disturbance | |
| BRAF inhibitors | Vemurafenib; | Decrease GFR; AKI; | Unclear |
| | Dabrafenib; | Glomerulonephritis; | |
| | Trametinib; | Hyponatremia; | |
| | Cobimetinib | Hypertension | |
| MTOR inhibitors | Temsirolimus | Glomerulopathy; AKI; | Unclear |
| | | Proteinuria | |
| BCR-ABL1 and | Bosutinib; Dasatinib; | Hypophosphatemia; | Rhabdomyolysis; |
| KIT inhibitors | Imatinib | Decreased GFR; AKI; | Thrombotic |
| | | Proteinuria; Nephrotic | thrombocytopeni |
| | | syndrome; CKD | purpura; Alterations in |
| | | | glomerular podocytes; |
| | | | Tumor lysis syndrome; |
| | | | Acute tubular injury |
| Anti-angiogenesis | Bevacizumab; | Hypertension; | Endothelial cell |
| drugs (VEGF | Ramucirumab; | Proteinuria; Nephrotic | dysfunction and |
| pathway inhibitors | Aflibercept; | syndrome; Decreased | dysregulation of |
| and TKI) | Sunitinib; Sorafenib; | GFR; TMA; | podocyte |
| | Pazopanib; | Glomerulopathy; | |
| | Ponatinib; Others | Electrolyte disturbance | |
| Inhibitor of Bruton's | Ibrutinib | AKI | Unclear, but tumor |
| tyrosine kinase | | | lysis syndrome might |
| | | | be contributory |
| Immune checkpoint | Ipilimumab; | Acute tubulointerstitial | Unclear, but |
| inhibitors (PD-1, | Pembrolizumab; | nephritis; Immune | development of |
| PD-L1, CTLA-4) | Nivolumab | complex | autoantibodies that are |
| | | glomerulonephritis; | pathogenic to the |
| | | TMA; Electrolyte | kidney might be |
| | | disturbance; AKI | contributory |
| | | (rare) | |
| Cytokine | INF-a | Proteinuria; | Minimal change |
| | | Glomerulopathy; | disease or focal |
| | | TMA; AKI | segmental |
| | | | glomerulosclerosis |
| Cytokine | IL-2 | AKI | Capillary leak |
| | | | syndrome leading to |
| | | | AKI |
| Peptide receptor | Lutetium Lu-177 | Decreased GFR | Kidney irradiation |
| radioligand | dotatate | | |

AKI: Acute kidney injury; CKD: Chronic kidney disease; GFR: Glomerular filtration rate; SIADH: Syndrome of inappropriate antidiuretic hormone secretion; TKI: Tyrosine kinase inhibitor; TMA: Thrombotic microangiopathy.

### Conclusion

One dose of CisPlatin is nephrotoxic as shown by the leakage of the biomarker osteopontin into the urine. One dose of FPS (8 mg per kg) administered intravenously and prophylactically can mitigate the harm caused by CisPlatin. The data suggested that repeated doses of FPS, given with a starting date before the administration of the nephrotoxic agent, and covering the period after the dose of the nephrotoxic agent's administration, would be helpful in the mitigation of the harm and can promote the recovery from the harm done to the kidney.

In this disclosure the word “harm” can mean “harm leading to physiological dysfunction, not necessarily caused by permanent damage” as well as “harm caused by structural damage in the kidney, mainly detected by OPN spillage into the urine.” The data showed that one dose of FPS given days before the administration of Cisplatin can protect the kidney up to and possibly beyond day 5, even though some harm in terms of dysfunction can be shown by serum BUN level as early as day 3.

According to one aspect, the present technology can include a method of treating renal failure, dysfunction or damage in a subject in need thereof. The method can include administering a therapeutically effective amount of an albumin nanoparticle suspension containing fibrinogen coated albumin spheres (FAS) into the subject to increase the concentration of stem cells or precursors cells in their original site and/or augment a function or effectiveness of stem cells or precursor cells in vivo to mobilize these stem cells or the precursor cells toward a trauma site.

According to another aspect, the present technology can include a method of prophylactically treating renal failure, dysfunction or damage in a subject caused by administration of a renal toxic agent or chemotherapy drug. The method can include administering, prior to administration of the renal toxic agent or the chemotherapy drug, a therapeutically effective amount of an albumin nanoparticle suspension containing fibrinogen coated albumin spheres (FAS) into the subject to increase the concentration of stem cells or precursors cells in their original site and/or augment a function or effectiveness of stem cells or precursor cells in vivo to mobilize these stem cells or the precursor cells toward a trauma site to mitigate harmful effects from the renal toxic agent or the chemotherapy drug.

According to still another aspect, the present technology can include an albumin nanoparticle suspension used for treating renal failure, dysfunction or damage in a subject in need thereof. The suspension can contain fibrinogen coated albumin spheres (FAS), and it can be configured to augment a function or effectiveness of stem cells or precursor cells in vivo to stimulate mobilization of stem cells or precursor cells toward a trauma site. A therapeutically effective amount of the suspension can be configured or configurable for intravenous administration into the subject.

In some embodiments, the renal failure, dysfunction or damage can be caused by administration to the subject of any one of or any combination of a renal-harmful agent, and a chemotherapy drug.

In some embodiments, the chemotherapy drug can contain platinum or can be CisPlatin.

In some embodiments, the chemotherapy drug is selected from the group consisting of Carboplatin (Paraplatin, Paraplatin AQ), Ifosfamide (Ifex), Methotrexate, Paclitaxel, Doxorubicin, and Gemcitabine.

In some embodiments, the therapeutically effective amount can be administered to the subject prophylactically and prior to administration of the renal-harmful agent or the chemotherapy drug.

In some embodiments, the stem cells or the precursor cells can be CD34+ stem cells.

In some embodiments, the fibrinogen coated albumin spheres can be configured to augment a function or an effectiveness of the CD34+ stem cells in vivo to stimulate mobilization of the CD34+ stem cells toward a site of injury.

In some embodiments, the fibrinogen coated albumin spheres can be configured to increase cell populations of the CD34+ stem cell inside any one of or any combination of bone marrow, kidney, and a muscle.

In some embodiments, the fibrinogen coated albumin spheres can be configured to increase in cell populations positive for CD34 cell markers or negative for CD34 cell markers.

In some embodiments, the fibrinogen coated albumin spheres can be configured to produce a favorable anti-oxidative status within an injured tissue of the subject leading to accelerated recovery of the injured tissue.

In some embodiments, the fibrinogen coated albumin spheres can be configured to accelerate a rate of improvement of histological structures inside a kidney or injured muscles of the subject, leading to improved function of the kidney or the muscles.

In some embodiments, the fibrinogen coated albumin spheres can be configured to produce biological molecules or vesicles that have paracrine effects, leading to an accelerated rate of improvement of renal function or of histological structures inside a kidney or injured muscles of the subject.

In some embodiments, the subject can be human.

In some embodiments, the therapeutically effective amount can be 4 mg/kg or more administered to the subject intravenously.

In some embodiments, the therapeutically effective amount can be 8 mg/kg or more administered to the subject intravenously.

In some embodiments, the therapeutically effective amount can be administered to the subject prophylactically and prior to the renal failure, dysfunction or damage.

Some embodiments of the present technology can include a step of testing for one or more biological markers in urine of the subject.

In some embodiments, the one or more biological markers can be osteopontin.

In some embodiments, a concentration amount of the one or more biological markers can be configured to predict a dose of the albumin nanoparticle suspension which is effective in a further treatment of renal failure, dysfunction or damage in the subject.

In some embodiments, the renal failure, dysfunction or damage can be selected from the group consisting of diabetic renal failures, Autosomal Dominant Polycystic Kidney Disease (ADPKD), and Minimal Change Disease (MCD).

### SUMMARY

This present technology involves the administration of fibrinogen-coated albumin spheres (FAS) in an animal model or human patients in the future with acute renal failure. A dose of 4 or more mg per kg, administered intravenously before or soon after onset of acute renal failure, was effective in the mitigation of the harm from renal-toxic agents. The mechanism of improvement is due possibly to (a) mobilization of stem cells or progenitor cells to the site of injury, and in addition, (b) paracrine effect of rescue cells at the site(s) needing healing, (c) suppression of the toxic effects of the renal-toxic agents, (d) suppression of pro-inflammatory cytokines, resulting in less leakage of renal markers into the urine, improved blood chemistry and histology of the previously injured site(s). It is likely that FAS can also be medically useful in chronic renal failures and in other non-kidney diseases that can benefit from the mechanisms of actions of FAS.

While embodiments of the protein nanospheres to treat renal failure, dysfunction or damage have been described in detail, it should be apparent that modifications and variations thereto are possible, all of which fall within the true spirit and scope of the present technology. With respect to the above description then, it is to be realized that the optimum administration, implementation, size, materials, shape, form, function and manner of operation of the present technology are deemed readily apparent and obvious to one skilled in the art, and all equivalent relationships to those illustrated in the drawings and described in the specification are intended to be encompassed by the present technology.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

What is claimed as being new and desired to be protected by Letters Patent of the United States is as follows:

1. A method of treating renal failure, dysfunction or damage in a subject in need thereof, the method comprising administering a therapeutically effective amount of an albumin nanoparticle suspension containing fibrinogen coated albumin spheres (FAS) into the subject to any one of or any combination of increase a concentration of stem cells or precursors cells in an original site thereof, and augment a function or effectiveness of the stem cells or precursor cells in vivo to mobilize the stem cells or the precursor cells toward a trauma site.

2. The method of claim 1, wherein the renal failure, dysfunction or damage is caused by administration to the subject of any one of or any combination of a renal-harmful agent, and a chemotherapy drug.

3. The method of claim 2, wherein the chemotherapy drug is selected from the group consisting of any platinum containing chemotherapy agent, CisPlatin, Carboplatin, Ifosfamide, Methotrexate, Paclitaxel, Doxorubicin, and Gemcitabine.

4. The method of claim 2, wherein the therapeutically effective amount is administered to the subject prophylactically and prior to administration of the renal-harmful agent or the chemotherapy drug.

5. The method of claim 1, wherein the stem cells or the precursor cells are CD34+ stem cells.

6. The method of claim 5, wherein the fibrinogen coated albumin spheres are configured to one of or any combination of increase the concentration of the CD34+ stem cells in an original site thereof, and augment a function or effectiveness of the CD34+ stem cells in vivo to mobilize the CD34+ stem cells toward a site of injury.

7. The method of claim 5, wherein the fibrinogen coated albumin spheres are configured to increase cell populations of the CD34+ stem cell inside any one of or any combination of bone marrow, kidney, and a muscle.

8. The method of claim 5, wherein the fibrinogen coated albumin spheres are configured to increase in cell populations positive for CD34 cell markers or negative for CD34 cell markers.

9. The method of claim 5, wherein the fibrinogen coated albumin spheres are configured to produce a favorable anti-oxidative status within an injured tissue of the subject leading to accelerated recovery of the injured tissue.

10. The method of claim 5, wherein the fibrinogen coated albumin spheres are configured to accelerate a rate of improvement of histological structures inside a kidney or injured muscles of the subject, leading to improved function of the kidney or the muscles.

11. The method of claim 5, wherein the fibrinogen coated albumin spheres are configured to produce biological molecules or vesicles that have paracrine effects, leading to an accelerated rate of improvement of renal function or of histological structures inside a kidney or injured muscles of the subject.

12. The method of claim 1, wherein the subject is human.

13. The method of claim 1, wherein the therapeutically effective amount is 4 mg/kg or more administered to the subject intravenously.

14. The method of claim 1, wherein the therapeutically effective amount is 8 mg/kg or more administered to the subject intravenously.

15. The method of claim 1, wherein the therapeutically effective amount is administered to the subject prophylactically and prior to the renal failure, dysfunction or damage.

16. The method of claim 1 further comprises the step of testing for one or more biological markers in urine of the subject.

17. The method of claim 16, wherein the one or more biological markers is osteopontin.

18. The method of claim 16, wherein a concentration amount of the one or more biological markers are configured to predict a dose of the albumin nanoparticle suspension which is effective in a further treatment of renal failure, dysfunction or damage in the subject.

19. The method of claim 16, wherein the renal failure, dysfunction or damage is selected from the group consisting of diabetic renal failures, Autosomal Dominant Polycystic Kidney Disease (ADPKD), and Minimal Change Disease (MCD).

20. A method of prophylactically treating renal failure, dysfunction or damage in a subject caused by administration of a renal toxic agent or chemotherapy drug, the method comprising administering, prior to administration of the renal toxic agent or the chemotherapy drug, a therapeutically effective amount of an albumin nanoparticle suspension containing fibrinogen coated albumin spheres (FAS) into the subject to any one of or any combination of increase a concentration of stem cells or precursors cells in an original site thereof, and augment a function or effectiveness of the stem cells or precursor cells in vivo to mobilize the stem cells or the precursor cells toward a trauma site to mitigate harmful effects from the renal toxic agent or the chemotherapy drug.
